(19) United States
(12) Patent Application Publication
(10) Pub. No.: US 2003/0050349 A1
(43) Pub. Date: Mar. 13, 2003

(54) In-situ desulfurization of a feed stream in a catalytic reactor

(75) Inventor: Joe D. Allison, Ponca City, OK (US)

Correspondence Address: DAVID W. WESTPHAL, CONOCO PHILLIPS, P.O. BOX 1267, PONCA CITY, OK, 74602-1267, US

(73) Assignee: Conoco Inc., Houston, TX (US)

(21) Appl. No.: 10/155,511

(22) Filed: May 24, 2002

Related U.S. Application Data
(60) Provisional application No. 60/316,673, filed on Aug. 31, 2001.

(51) Int. Cl. C07C 27/06; B01J 27/24

(52) U.S. Cl. 518/715; 502/200

(57) ABSTRACT

The present invention relates to a method for removing a sulfur containing catalyst poison from a feedstock. Benefits from removing catalyst poisoning sulfur compounds in a feedstock include upgrading the quality of the various petroleum fractions and prolonging the life of the catalyst. A preferred embodiment of the present invention includes adding a sacrificial metal to a Fischer-Tropsch reactor. The role of the sacrificial metal is adsorption of the sulfur-containing species that may deactivate or poison the catalyst.

### CROSS-REFERENCE TO RELATED APPLICATIONS

The present application claims the benefit of 35 U.S.C. 111(b) provisional application Serial No. 60/316,673 filed Aug. 31, 2001, and entitled In-Situ Desulfurization of a Feed Stream in a Catalytic Reactor.

### FIELD OF THE INVENTION

The present invention generally relates to a method for removing a poison from a feedstock. More particularly, the present invention relates to a method for extending the life of a catalyst, preferably a Fischer-Tropsch catalyst, more preferably a cobalt-containing Fischer-Tropsch catalyst. Still more particularly, the present invention relates to a method of removing a sulfur from a feedstock that includes adding a sacrificial metal to a reactor, the sacrificial metal preferably having an affinity for sulfur, more preferably binding with sulfur.

### BACKGROUND OF THE INVENTION

Large quantities of methane, the main component of natural gas, are available in many areas of the world. Methane can be used as a starting material for the production of hydrocarbons. The conversion of methane to hydrocarbons is typically carried out in two steps. In the first step methane is reformed with water or partially oxidized with oxygen to produce carbon monoxide and hydrogen (i.e., synthesis gas or syngas). In a second step, the syngas is converted to hydrocarbons.

This second step, the preparation of hydrocarbons from synthesis gas, is well known in the art and is usually referred to as Fischer-Tropsch synthesis, the Fischer-Tropsch process, or Fischer-Tropsch reaction(s). The Fischer-Tropsch synthesis is typically carried out with the aid of a catalyst. A catalyst desirably denotes a compound that increases the rate of a reaction without itself being consumed by the reaction. Catalysts for use in the Fischer-Tropsch synthesis usually contain a catalytic metal of Groups 8, 9, 10 (in the New notation of the periodic table of the elements, which is followed throughout). In particular, iron, cobalt, nickel, and ruthenium have been abundantly used as the catalytic metals. Cobalt and ruthenium have been found to be active for catalyzing a process in which synthesis gas is converted to primarily hydrocarbons having five or more carbon atoms (i.e., where the C₅₊ selectivity of the catalyst is high).

Many times poisons are carried into reactors with the feed. When poisons come in contact with catalysts, they can lower the rates for desired reactions or promote undesirable reactions. More particularly, sulfur species in a process feed, which may an be inorganic compound (e.g. H₂S, COS) or an organic compound (e.g. RSH), will often combine with the catalytic metals used and form their respective metal sulfides. Because the catalytic metals used in the conversion of methane to hydrocarbons have a high affinity for sulfur, this usually poisons the catalyst such that the desired reaction no longer occurs. While this is detrimental to the efficiency of the reactor, inasmuch as poisoned catalyst must be replaced or rehabilitated, it is also very costly.

A typical supported metal catalyst may cost in the range of $20-$40 per pound, of which the cost of the metals may be between 50-80%. Thus, for a reactor that uses 2 million pounds of catalyst, the total cost of the metals is considerable. Further, the catalyst is designed to last a few years, after which time it is replaced. Premature shutdowns due to the death of a catalyst are always expensive and the cost of replacement catalyst is considerable.

In many situations it is desirable to pre-treat the feed stream before it comes in contact with the catalyst. Many conventional pretreatment methods include extracting elemental sulfur from H₂S or gaseous mixtures containing moderate to high concentrations of H₂S. Reducing the sulfur content of the feed stream prolongs the life of the catalyst and upgrades the quality of the various petroleum fractions produced in the reactor.

As described above, the usual approach taken to overcome these problems is to desulfurize the feedstock before it contacts the catalyst. The broad approach taken in order to accomplish this goal usually involves hydrosulfurization and absorption of the resultant H₂S. See for example U.S. Pat. No. 5,738,834, incorporated herein by reference. The stream is then treated with a variety of desulfurization agents before entering the catalytic reactor. Exemplary desulfurization agents include copper-zinc and copper-zinc-aluminum, zinc and/or zinc oxide mixed with nickel and/or nickel oxide, zinc oxide followed by nickel, and molecular sieve followed by zinc oxide or a bimetallic spinel. Some of these processes can be economically unfeasible, while others do not remove the sulfur-containing contaminants to a sufficiently low value. Further, a disadvantage of conventional methods of desulfurizing the feedstock before it contacts the catalyst is the occasional breakthrough of sufficient sulfur to poison the catalyst.

Two alternative approaches have also received some attention. The catalyst itself may be made more resistant to poisoning. U.S. Pat. No. 4,101,451 describes the impregnation of a copper catalyst with hydroxide followed by heating for chemically increased resistance to sulfur poisoning. Alternatively, U.S. Pat. No. 5,928,980 describes the use of a spent catalyst contaminated with heavy metals as a support impregnated with new catalyst for a different system. The contamination helps to absorb the sulfur from the feedstock.

However, these approaches tend to be costly, time-intensive and to have various limitations and requirements. Hence, a need exists for a cost-effective, timely method of in situ reduction of sulfur poisoning of Fischer-Tropsch catalysts.

### SUMMARY OF THE INVENTION

The present invention overcomes the deficiencies of the prior art by providing a method in which concurrent hydrocarbon conversion and desulfurization of a feedstock occur by including in the catalytic process a sacrificial metal species with the capability to adsorb the sulfur-containing species chemically. The sacrificial metal is preferably present together with a catalytic metal on a support. Alternatively, the sacrificial metal may be separately present in the reactor. Sacrificial, defined herein, refers to a metal with a higher affinity for sulfur than the catalytic metal. More particularly, a sacrificial metal is preferably a metal for which a compound containing the sacrificial metal and sulfur has a pKₛₚ greater than the pKₛₚ of a compound containing a catalytic metal and sulfur. Thus, when the catalytic metal is cobalt, a sacrificial metal is preferably a metal for which a compound containing the sacrificial metal and sulfur has a pKₛₚ of at least 25. As is conventional, the ‘p’ stands for the negative log (base 10) of the number. The larger a pKₛₚ of a compound, the less soluble the compound is in water. The present method is used preferably in addition to a conventional sulfur removal technique such as zinc bed absorption columns. Further, the entering sulfur content in the feed stream is preferably less than 10 ppm, more preferably less than 1 ppm, still more preferably less than 0.1 ppm.

In accordance with an embodiment of the present invention, a method for extending the life of a catalyst includes selecting a sacrificial metal having a poison affinity at least equal to a predetermined poison affinity, providing a catalyst material having a sacrificial metal and a catalytic metal, and contacting a poison-containing feed stream with the catalyst material.

According to an alternate embodiment of the present invention, a process for producing hydrocarbons includes contacting a feed stream of hydrogen and carbon monoxide with a catalyst system in a reaction zone maintained at conversion-promoting conditions effective to produce an effluent stream of hydrocarbons. The catalyst system preferably includes a catalytic metal and a sacrificial metal.

According to another preferred embodiment of the invention, a catalyst system for sulfur removal in a Fisher-Tropsch feed stream includes a catalyst system having a sacrificial metal and a catalytic metal. The sacrificial metal preferably has a sulfur affinity at least equal to the catalytic metal's sulfur affinity.

### DETAILED DESCRIPTION OF THE PREFERRED EMBODIMENT

The present invention entails the use of a catalyst material that includes a combination of a sacrificial metal and a catalytic metal. By preferentially complexing with a poison in a process feed, the sacrificial metal acts to reduce poisoning of the catalytic metal by the poison. This has the advantage of prolonging catalyst life and reducing reactor down time.

According to one embodiment of the present invention, a sacrificial metal and a catalytic metal are present in an intimate mixture. For example, they may be deposited together on a catalyst support via methods known by one of ordinary skill in the art (i.e. melt impregnation). Thus, the supported material may include the catalytic metal and the sacrificial metal, co-supported on the catalyst support.

According to an alternative embodiment of the present invention, the sacrificial metal and the catalytic metal are present in a physical mixture. For example, the catalytic metal may be supported on a catalyst support and the sacrificial metal on an additional support via methods known by one of ordinary skill in the art (i.e. melt impregnation).

Either or both of the catalyst or any separate sacrificial metal component may be in skeletal form. That is, either or both of the catalytic metal and the sacrificial metal may form the structure of the catalyst as well as the catalytically active component.

The sacrificial metal is preferably chosen such that it has a higher affinity for the poison than the affinity of a catalytic metal for the poison. More particularly, the sacrificial metal preferably chemically binds to the poison more strongly than the catalytic metal, thus reducing species containing the poison bound to the catalytic metal. Measures of binding include solubility product of the formed metal species.

An advantage of the stronger bonding of the poison to the sacrificial metal is that even if the poison is initially bound to catalytic metal, some small equilibrium between free and bound poison is likely. This equilibrium frees some of the poison to bind with the sacrificial metal. The smaller equilibrium constant of the sulfide of the sacrificial metal, as compared to the equilibrium constant of the sulfide of the catalytic metal drives the overall binding to the sacrificial metal.

Catalyst

The selection of a catalyst requires many technical and economic considerations. The process of selecting a precious metal catalyst can be broken down into components. Desirable catalyst properties include high activity, high selectivity, high recycle capability and filterability. Catalyst performance is determined mainly by the precious metal component. A metal is chosen based both on its ability to complete the desired reaction and its inability to complete an unwanted reaction.

Typically, a catalytic metal is supported by a matrix material. A catalyst support is any of a variety of essentially inactive materials on which a catalytically active material can be coated. In general, a catalyst support should allow for a high degree of metal dispersion. The choice of support is largely determined by the nature of the reaction system. A support should be stable under reaction and regeneration conditions, and not adversely react with solvent, reactants, or reaction products. Common powdered supports include activated carbon, alumina, silica, silica-alumina, carbon black, TiO2, ZrO2, CaCO3, and BaSO4. The majority of precious metal catalysts are supported on either carbon or alumina. The present catalyst material may be supported on any suitable support. Supports that are contemplated for use with a catalyst according the preferred embodiments of the present invention include silica, titania, titania/alumina, zirconia, alumina, silica, titania, titania/alumina, and the like. Further, suitable supports include those disclosed in commonly assigned U.S. Pat. No. 6,368,997, issued from U.S. application Ser. No. 09/314,921, entitled “Fischer-Tropsch Catalysts and Processes Using Fluorided Supports; U.S. Pat. No. 6,365,544, issued from U.S. application Ser. No. 09/314,920, entitled “Fischer-Tropsch Processes and Catalysts Using Fluorided Alumina Supports”, and co-pending U.S. application Ser. No. 09/898,287, entitled “Fischer-Tropsch Processes and Catalysts Using Aluminum Borate Supports”, each hereby incorporated herein by reference. Thus, suitable supports further may include fluorided metal oxides, fluorided alumina, aluminum fluoride, borated alumina, and aluminum borate.

A support can affect catalyst activity, selectivity, recycling, refining, material handling and reproducibility. Critical properties of a support include surface area, pore volume, pore size, distribution, particle size, attrition resistance, acidity, impurity levels, and the ability to promote metal-support interactions. Metal dispersion increases with surface support area. Support porosity affects metal dispersion and distribution, metal sintering resistance, and intraparticle diffusion of reactants, products and poisons. Smaller support particle size increases catalytic activity but decreases filterability. A support should have desirable mechanical properties, including attrition resistance and hardness. An attrition resistant support allows improved catalyst recycling and rapid filtration. Support impurities may deactivate the metal and enhance catalyst selectivity.

In a preferred embodiment of the present invention, the catalytic metal is selected from the group including Group 8 elements, Group 9 elements, and Group 10 elements. As described above, catalysts for use in the Fischer-Tropsch synthesis usually contain a catalytic metal of Groups 8, 9, 10.

In one embodiment of the present invention, the catalytic metal includes cobalt. Cobalt is preferably present in the catalyst material in an amount catalytically effective for the Fischer-Tropsch reaction. The amount of cobalt present in the catalyst may vary widely. Typically, the catalyst includes from about 10 to about 40 wt. % cobalt, more preferably from about 15 to about 30 wt. % cobalt, most preferably from about 18 to about 22 wt. % cobalt.

It will be understood that any suitable promoter that does not interact with the sacrificial metal to inhibit its affinity for sulfur may be included in the catalytic material. The promoter is preferably a promoter for a Fischer-Tropsch catalyst, more preferably a cobalt-based catalyst. The promoter may be any known Fischer-Tropsch promoter, preferably one that improves the activity of a catalyst in the Fischer-Tropsch reaction. The promoter is preferably selected from the group consisting of the elements of Group 5 (e.g. V, Nb, and Ta), the elements of Group 6 (e.g. Cr, Mo, and W), the elements of Group 7 (e.g. Mn, Tc, and Re), the elements of Group 8, the elements of Group 9, the elements of Group 10, the elements of Group 11 (e.g. Cu, Ag, and Au), the elements of Group 12 (e.g. Zn, Cd, and Hg), the elements of Group 13 (e.g. B, Al, Ga, In, and Tl), and the elements of Group 14 (e.g. C, Si, Ge, Sn, and Pb) of the Periodic Table, more preferably from among rhenium, ruthenium, platinum, copper, silver, boron, manganese, still more preferably from among boron, copper, platinum, and silver.. When the catalytic material includes a promoter, the promoter is preferably added to a cobalt-containing catalyst in an amount to provide a ratio of elemental cobalt to elemental promoter of about 2 to 1, more preferably from about 20 to about 1, most preferably from about 200 to about 1. Further, suitable promoters, and concentrations thereof, include those disclosed in commonly assigned co-pending U.S. Pat. No. 6,333,294 issued from U.S. application Ser. No. 09/314,811, entitled “Fischer-Tropsch Processes and Catalysts Using Promoters”, and U.S. utility application Ser. No. 09/804,271, entitled “Fischer-Tropsch Processes and Catalysts with Promoters”, and U.S. utility application Ser. No. 10/047,231, entitled “Boron Promoted Catalysts and Fischer-Tropsch Processes”, and U.S. Provisional Application Serial No. 60/316,826, Attorney Docket Number 1856-14400, entitled “Fischer-Tropsch Processes and Catalyst Using Promoters”, each hereby incorporated herein by reference.

Sacrificial Metal

The choice of sacrificial metal preferably follows from the choice of catalytic metal. The sacrificial metal preferably complexes with sulfur. Sulfur is a known poison for iron-containing Fischer-Tropsch catalysts and cobalt-containing Fischer-Tropsch catalysts.

A measure of the relative affinity of a sacrificial metal and of a catalytic metal for sulfur may be obtained by comparing, for example, the Kₛₚ of their respective sulfur compounds. Kₛₚ is the conventional solubility product.

When the catalytic metal is cobalt, suitable sacrificial metals include, but are not limited to bismuth, indium, mercury, tungsten, thallium, calcium, copper, magnesium, silver, tin, antimony, cadmium, lead, molybdenum, tungsten, and combinations thereof. Vaues of a pKₛₚ for sulfur compounds of these sacrificial metals, as well as for cobalt, are listed in Table 1, for temperatures near room temperature, that is between 18° C. and 25° C. As is conventional, pKₛₚ=−log(Kₛₚ), where log indicates the logarithm base 10. Where a range is given in Table 1, reported values are within that range, as reported in one or more of the following references: Handbook of Chemistry and Physics, published by the Chemical Rubber Company Press, 62^{nd} edition, page B-242; “General Chemistry: An Integrated Approach”, by Hill and Petrucci, published by Prentice-Hall, 2^{nd} edition, Chapter 16, also at http://www.cw.prenhall.com/bookbind/pubbooks/hill2/medialib/tools/solubility.html as displayed on Jul. 6, 2001; http://www.chem.ualberta.ca/courses/plambeck/p101/new/p00407.htm as displayed on Jul. 6, 2001.; http://www2.austin.cc.tx.us/rvsmthsc/chem/chem-Solubili-2.html as displayed on or before Jul. 6, 2001.; http://www.ktf-split.hr/periodni/en/abcd/kpt.html as displayed on Jul. 6, 2001.; and http://bilbo.chm.uri.edu/CHM112/tables/KspTable.htm as displayed on Jun. 29, 2001 each hereby incorporated herein by reference.

TABLE 1

| Metal | Compound | pKₛₚ |
|---|---|---|
| Cobalt | CoS | 20-25 |
| Iron | FeS | 17-18 |
| Nickel | NiS | 24-26 |
| Cadmium | CdS | 26-29 |
| Copper (II) | CuS | 35-36 |
| Copper (I) | Cu₂S | 47-48 |
| Lead | PbS | 27-28 |
| Bismuth | Bi₂S₃ | 97 |
| Indium | In₂S₃ | 73 |
| Mercury (I) | Hg₂S | 46-47 |
| Mercury (II) | HgS | 52-53 |
| Silver | Ag₂S | 49-50 |
| Antimony | Sb₂S₃ | 93 |
| Tin | SnS | 25-27 |
| Zinc | ZnS | 22-25 |

The sacrificial metal is preferably added to a cobalt-containing catalyst in an amount to provide a ratio of elemental cobalt to elemental sacrificial metal of about 2 to 1, more preferably from about 20 to about 1, most preferably from about 200 to about 1.

When the sacrificial metal is supported separately from the catalytic metal, the support for the sacrificial metal may be selected from among supports suitable as catalyst supports.

Suitable supports for the sacrificial metal include any one of the supports for catalytic metal described above. Thus, for example, sutiable supports include silica, titania, titania/alumina, zirconia, alumina, aluminum fluoride, and fluorided alumina, borated alumina, silica, titania, and titania/alumina, and the like.

Preparation of Catalyst Material

The most preferred method of catalyst preparation may vary among those skilled in the art, depending for example on the desired catalyst particle size. Those skilled in the art are able to select the most suitable method for a given set of requirements.

A preferred method of preparing a supported metal catalyst is by incipient wetness impregnation of the support with an aqueous solution of a soluble metal salt such as nitrate, acetate, acetylacetonate or the like. Another method of preparing a supported metal catalyst is by a melt impregnation technique, which involves preparing the supported metal catalyst from a molten metal salt. Further, the catalyst may be prepared using a surfactant, as described in commonly assigned co-pending provisional application entitled “Surface Active Agent Use in Catalyst Preparation,” Attorney Reference Number 1856-08300, filed Jul. 3, 2001, hereby incorporated herein by reference.

When the catalytic metal includes cobalt, one method is to impregnate the support with a molten metal nitrate (e.g., Co(NO₃)₂.6H₂O). Alternatively, the support can be impregnated with a solution of zero valent metal precursor. A method is to impregnate the support with a solution of zero valent cobalt such as Co₂(CO)₈, Co₄(CO)₁₂ or the like in a suitable organic solvent (e.g., toluene).

The sacrificial metal, precursor compound may be dissolved in a suitable solvent, e.g. water mixed with the cobalt precursor and impregnated with the cobalt on the support. Alternatively, the sacrificial method may be dissolved in a solvent and impregnated on the support either before or after the cobalt is impregnated. Still alternatively, the sacrificial metal precursor may be dissolved in a solvent and impregnated on a separate support.

The impregnated support is treated, yielding a prepared catalyst that may be stored, preferably in an inert environment, until the catalyst is used. The treatment preferably includes drying the impregnated support, followed by optional moisture absorption. In one preferred method, the impregnated support is dried and reduced with hydrogen or a hydrogen containing gas. In another preferred method, the impregnated support is dried, oxidized with air or oxygen and reduced in the presence of hydrogen. The hydrogen reduction step may not be necessary if the catalyst is prepared with zero valent cobalt. Typically, at least a portion of the metal(s) of the catalytic metal component of the catalysts of the present invention is present in a reduced state (i.e., in the metallic state). Therefore, it is normally advantageous to activate the catalyst in situ prior to use by a reduction treatment, in the presence of hydrogen at an elevated temperature. Thus, this in situ reduction may be a second reduction step, additional to the optional reduction described above. Typically, in situ reduction of the catalyst includes treating the catalyst with hydrogen at a temperature in the range of from about 75° C. to about 500° C., for about 0.5 to about 24 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as carbon monoxide and carbon dioxide. Reduction with pure hydrogen and reduction with a mixture of hydrogen and carbon monoxide are preferred. The amount of hydrogen may range from about 1% to about 100% by volume. The hydrogen reduction step may not be necessary if the catalyst is prepared with zero valent cobalt.

Fischer-Tropsch Process

The catalyst material containing a sacrificial metal and a catalytic metal according to an embodiment of the present invention is preferably used in a catalytic process for production of hydrocarbons, most preferably the Fischer-Tropsch process. The feed gases charged to the process of a preferred embodiment of the present invention comprise hydrogen, or a hydrogen source, and carbon monoxide. H₂/CO mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, the feed gas stream contains hydrogen and carbon monoxide in a molar ratio of about 2:1. The feed gas may also contain carbon dioxide. The feed gas stream should contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, ammonia and carbonyl sulfides. In a preferred embodiment, the entering sulfur content in the feed stream is preferably less than 10 ppm, more preferably less than 1 ppm, still more preferably less than 0.1 ppm.

The feed gas is contacted with the catalyst material in a reaction zone. Mechanical arrangements of conventional design may be employed as the reaction zone including, for example, plugged flow, continuous stirred tank, fixed bed, fluidized bed, slurry phase, slurry bubble column, reactive distillation column, or ebulliating bed reactors, among others, may be used. A slurry bubble column reactor is described in U.S. Pat. No. 4,429,159, hereby incorporated herein by reference. Plug flow, fluidized bed, reactive distillation, ebulliating bed, and continuous stirred tank reactors have been delineated in “Chemical Reaction Engineering,” by Octave Levenspiel, and are known in the art. The size and physical form of the catalyst may vary, depending on the reactor in which it is to be used.

When the feed gas contacts the catalyst material, a portion of the poison preferably binds to the sacrificial metal. In a preferred embodiment, at least 50 wt % of the poison binds to the sacrificial metal, reducing the poison content in the effluent stream. After the feed gas contacts the catalyst material, the effluent stream preferably comprises less than 5 ppm poison.

The Fischer-Tropsch process is typically run in a continuous mode. In this mode, the gas hourly space velocity through the reaction zone typically may range from about 100 volumes/hour/volume catalyst (v/hr/v) to about 10,000 v/hr/v, preferably from about 300 v/hr/v to about 2,000 v/hr/v. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psig (653 kPa) to about 1000 psig (6994 kPa), preferably, from 80 psig (653 kPa) to about 600 psig (4237 kPa), and still more preferably, from about 140 psig (1066 kPa) to about 400 psig (2858 kPa).

The products resulting from the process will have a great range of molecular weights. Typically, the carbon number range of the product hydrocarbons will start at methane and continue to the limits observable by modern analysis, about 50 to 100 carbons per molecule. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to effect the condensation of additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. While preferred embodiments of this invention have been shown and described, modifications thereof can be made by one skilled in the art without departing from the spirit or teaching of this invention. The embodiments described herein are exemplary only and are not limiting. Many variations and modifications of the catalyst and process are possible and are within the scope of the invention. Accordingly, the scope of protection is not limited to the embodiments described herein, but is only limited by the claims that follow, the scope of which shall include all equivalents of the subject matter of the claims.

What is claimed is:

1. A method for extending the life of a catalyst that includes a catalytic metal, the method comprising:
a) selecting a sacrificial metal having a poison affinity at least equal a predetermined poison affinity;
b) providing a catalyst material comprising the sacrificial metal and the catalytic metal; and
c) contacting a feed stream with the catalyst material, wherein the feed stream comprises the poison.

2. The method according to claim 1 wherein the poison comprises sulfur.

3. The method according to claim 2 wherein the feed stream comprises not more than 10 ppm sulfur.

4. The method according to claim 2 wherein a pKₛₚ of a sulfur compound of the sacrificial metal is at least equal to a predetermined pKₛₚ.

5. The method according to claim 4 wherein the predetermined pKₛₚ is about 25.

6. The method according to claim 2 wherein a pKₛₚ of a sulfur compound of the sacrificial metal is at least equal to a pKₛₚ of a sulfur compound of the catalytic metal.

7. The method according to claim 1 wherein at least a portion of the poison binds to the sacrificial metal.

8. The method according to claim 7 wherein at least 50 wt % of the poison binds to the sacrificial metal.

9. The method according to claim 1 wherein the sacrificial metal comprises a metal selected from the group consisting of bismuth, indium, mercury, thallium, calcium, copper, magnesium, silver, tin, antimony, cadmium, lead, molybdenum, tungsten, and combinations thereof.

10. The method according to claim 1 wherein the catalytic metal comprises a metal selected from the Group consisting of the elements of Group 8, the elements of Group 9, and the elements of Group 10.

11. The method according to claim 10 wherein the catalytic metal comprises cobalt.

12. The method according to claim 11 wherein the cobalt to sacrificial metal ratio is 2:1.

13. The method according to claim 1 wherein step (b) comprises mixing the sacrificial metal and the catalytic metal.

14. The method according to claim 13 wherein the step (b) comprises forming an intimate mixture of the sacrificial metal and the catalytic metal.

15. The method according to claim 14 wherein step (b) comprises impregnating the sacrificial metal and the catalytic metal on a support.

16. The method according to claim 13 wherein step (b) comprises forming a physical mixture of the sacrificial metal and the catalytic metal.

17. The method according to claim 16 wherein step (b) comprising impregnating the sacrificial metal supported on a first support and impregnating the catalytic metal on a second support.

18. A process for producing hydrocarbons, comprising contacting a feed stream comprising hydrogen and carbon monoxide with a catalyst system that includes a sacrificial metal and a catalytic metal in a reaction zone maintained at conversion-promoting conditions effective to produce an effluent stream of hydrocarbons.

19. The process according to claim 18 wherein the feed stream comprises a poison.

20. The process according to claim 19 wherein the poison comprises sulfur.

21. The process according to claim 20 wherein the feed stream comprises not more than 10 ppm sulfur.

22. The method according to claim 20 wherein a pKₛₚ of a sulfur compound of the sacrificial metal is at least equal to a predetermined pKₛₚ.

23. The method according to claim 20 wherein the predetermined pKₛₚ is about 25.

24. The method according to claim 20 wherein a pKₛₚ of a sulfur compound of the sacrificial metal is at least equal to a pKₛₚ of a sulfur compound of the catalytic metal.

25. The method according to claim 19 wherein at least a portion of the poison binds to the sacrificial metal.

26. The method according to claim 25 wherein the effluent stream comprises not more than 5 ppm poison.

27. The process according to claim 18 wherein the catalytic metal comprises a metal selected from the Group consisting of the elements of Group 8, the elements of Group 9, and the elements of Group 10.

28. The process according to claim 27 wherein the catalytic metal comprises cobalt.

29. The process according to claim 28 wherein the cobalt to sacrificial metal ratio is 2:1.

30. The process according to claim 18 wherein the sacrificial metal comprises a metal selected from the group consisting of bismuth, indium, mercury, thallium, calcium, copper, magnesium, silver, tin, antimony, cadmium, lead, molybdenum, tungsten, and combinations thereof.

31. The process according to claim 18 wherein the catalyst system comprises an intimate mixture of the sacrificial metal and catalytic metal.

32. The process according to claim 18 wherein the catalyst system comprises a physical mixture of the sacrificial metal and catalytic metal.

33. A catalyst system for sulfur removal in a Fisher-Tropsch feed stream comprising:
a catalyst system comprising a sacrificial metal and a catalytic metal, wherein said sacrificial metal has a sulfur affinity at least equal to the catalytic metal's sulfur affinity.

34. The catalyst system according to claim 33 wherein the sulfur affinity is measured by a pKₛₚ of a compound of sulfur and the sacrificial metal.

35. The catalyst system according to claim 33 wherein the feed stream comprises 10 ppm sulfur.

36. The catalyst system according to claim 33 wherein said sacrificial metal is adapted to bind to said sulfur.

37. The catalyst system according to claim 36 wherein said sacrificial metal is adapted to bind at least 50 wt % of said sulfur.

38. The catalyst system according to claim 33 wherein the catalytic metal comprises a metal selected from the Group consisting of the elements of Group 8, the elements of Group 9, and the elements of Group 10.

39. The catalyst system according to claim 38 wherein the catalytic metal comprises cobalt.

40. The catalyst system according to claim 39 wherein the cobalt to sacrificial metal ratio is 2:1.

41. The catalyst system according to claim 33 wherein the sacrificial metal comprises a metal selected from the group consisting of bismuth, indium, mercury, thallium, calcium, copper, magnesium, silver, tin, antimony, cadmium, lead, molybdenum, tungsten, and combinations thereof.

42. The catalyst system according to claim 33 wherein the catalyst system comprises an intimate mixture of the sacrificial metal and catalytic metal.

43. The catalyst system according to claim 33 wherein the catalyst system comprises a physical mixture of the sacrificial metal and catalytic metal.
